(19) United States
(12) Patent Application Publication
(10) Pub. No.: US 2005/0222637 A1
(43) Pub. Date: Oct. 6, 2005

(54) Tachygastrial electrical stimulation

(75) Inventor: Jiande Chen, Houston, TX (US)

Correspondence Address: FITCH EVEN TABIN AND FLANNERY, 120 SOUTH LA SALLE STREET, SUITE 1600, CHICAGO, IL, 60603-3406, US

(73) Assignee: Transneuronix, Inc.

(21) Appl. No.: 11/058,572

(22) Filed: Feb. 15, 2005

Related U.S. Application Data
(60) Provisional application No. 60/557,737, filed on Mar. 30, 2004.

(51) Int. Cl. A61N 1/18

(52) U.S. Cl. 607/40

(57) ABSTRACT

A process and device for treating obesity and syndromes related to motor disorders of the stomach of a patient is provided. The process includes artificially altering, by means of electrical pulses for preset periods of time, the natural gastric motility of the patient to prevent the emptying of or to slow down gastric transit through the stomach to increase the feeling of satiety and/or to accelerate intestinal transit to reduce absorption time within the intestinal tract. More specifically, the electrical stimulation induces tachygastria, which inhibits gastric motility, yields gastric distention, and delays gastric emptying. The tachygastrial electrical stimulation of the stomach, or other portions of the gastrointestinal tract, includes relatively long pulse widths, with lengths of up to 500 milliseconds.

### CROSS-REFERENCE TO RELATED APPLICATION

This application claims the benefit of U.S. Provisional application Ser. No. 60/557,737, filed Mar. 30, 2004, which is incorporated by reference in its entirety herein.

### FIELD OF THE INVENTION

The present invention relates to processes and to devices for treating obesity and syndromes related to motor disorders of the stomach and, more particularly, to processes and devices for treating obesity and syndromes related to motor disorders of the stomach with electrical stimulation of the gastrointestinal tract, wherein the electrical stimulation comprises tachygastrial electrical stimulation.

### BACKGROUND OF THE INVENTION

Patients having an excessively high amount of body fat or adipose tissue in relation to lean body mass are considered obese; such obese patients have a body mass index or BMI (i.e., the ratio of weight in kilograms to the square of the height in meters) of 30 kg/m² or more. Morbidly obese patients are generally defined to have a body mass index of greater than 40 kg/m². The adverse health effects of obesity, and more particularly morbid obesity, have become well-known in recent years. Such adverse health effects include, but are not limited to, cardio-vascular disease, diabetes, high blood pressure, arthritis, and sleep apnea. Generally, as a patient's body mass index rises, the likelihood of suffering the adverse health effects of obesity also rises.

Often, surgery has been the only therapy that ensures real results in patients whom have exceeded BMI values close to, or in excess of, 40 kg/m². Modern surgical procedures generally entail either (1) the reduction of gastric compliance, with the aim of limiting the subject's ability to ingest food, or (2) the reduction of the food absorption surface by shortening or bypassing part of the digestive canal. In some case, both aims are sought through the same surgical procedure. Since the major surgical procedures (e.g., removal or blocking off of a portion of the stomach) currently in use have some immediate and/or delayed risks, surgery is considered an extreme solution for use only when less invasive procedures fail. Furthermore, even surgical treatment fails in some cases, thereby requiring the surgeon to attempt to correct the problem or restore the original anatomical situation.

Recently, however, methods have been successfully employed whereby an electrical stimulation device is implanted on the stomach wall and/or small intestine. For example, U.S. Pat. No. 5,423,872 (Jun. 13, 1995) provides a process for the treatment of obesity and related disorders employing an electrical stimulator or pacemaker attached to the antrum or greater curvature of the stomach. U.S. Pat. No. 6,615,084 (Sep. 2, 2003) provides a process for the treatment of obesity and related disorder employing an electrical stimulator or pacemaker attached to the lesser curvature of the stomach. U.S. Pat. No. 5,690,691 (Nov. 25, 1997) provides a portable or implantable gastric pacemaker including multiple electrodes positionable on the inner or outer surface of an organ in the gastrointestinal tract which are individually programmed to deliver a phased electrical stimulation to pace peristaltic movement of material through the gastrointestinal tract. U.S. patent application Ser. No. 10/627,908 (filed Jul. 25, 2003) provides methods whereby an electrical stimulation device is implanted on the small intestines or lower bowel. More recently, U.S. Pat. No. 6,606,523 (Aug. 12, 2003) provides an apparatus for stimulating neuromuscular tissue of the gastrointestinal tract and methods for installing the apparatus to the surface of the neuromuscular tissue. Although these methods have generally been successful, it is still desirable to provide improved methods for such treatments. The present invention provides such an improved process.

In the treatment of obesity, electrical stimulation of the stomach delays the stomach transit and/or increases the patients feeling of “fullness,” thus decreasing the amount of food ingested, by continuous disruption of the intrinsic electrical activity during periods of therapy. Such continuous disruption may result in weight loss by decreasing stomach contractions, distending the stomach and thus inducing the feeling of satiety, changing the intrinsic direction and frequency of the peristalsis during periods of therapy, and/or modulating the sympathetic nervous system. Also in the treatment of obesity, electrical stimulation of the small intestine decreases the small intestinal transit time by efficient electrical induction of peristalsis thereby increasing the speed of material moving through the intestine and reducing the level of absorbed components.

### SUMMARY OF THE INVENTION

The present invention provides a process for treating obesity and/or related motor disorders by providing at least one electrostimulation or pacemaker device attached to, or adjacent to, the stomach and/or small intestines. The electrostimulation method of the present invention utilizes relatively long electrical pulse widths, with pulse widths of up to 500 milliseconds. The individual pulses are generally at a rate of about 2 to about 30 pulses/minute, with each pulse lasting between about 50 and about 500 milliseconds, such that there is a pause of about 3 to about 30 seconds between the pulses. More preferably, the individual pulses are at a rate which is at least 30 percent higher than the patient's normal gastric slow waves. Preferably, the pulse amplitude is about 1 to about 20 milliamperes.

The process of the present invention involves treatment of obesity and other syndromes related to motor disorders of the stomach of a patient. The process comprises artificially altering, using sequential electrical pulses for preset periods of time, the natural gastric motility of the patient to prevent or slow down stomach emptying, thereby slowing food transit through the digestive system. Although not wishing to be limited by theory, electrical stimulation of the stomach appears to result in an expansion of the stomach, a feeling of satiation, and reduced intake of food. Again not wishing to be limited by theory, it appears that the electrical stimulation of the stomach also delays transit of ingested food through the stomach, thus further increasing the satiety of the patient. More specifically, the process of the present invention induces tachygastria, an electrical disrhythmia of the stomach that is known to inhibit gastric motility, in order to artificially alter the natural gastric motility of the patient.

The present invention provides a tachygastrial electrical stimulation method for treatment of a motor disorder of a patient's stomach, the method comprising implanting at least one electrostimulation device comprising one or more electrostimulation leads and an electrical connector for attachment to a pulse generator such that the one or more electrostimulation leads are attached to, or adjacent to, the stomach, whereby electrical stimulation can be provided to the stomach through the one or more electrostimulation leads, and supplying electrical stimulation having long pulse widths of about 50 to about 500 milliseconds to the stomach through the one or more electrostimulation leads.

### BRIEF DESCRIPTION OF THE DRAWINGS

FIG. 1 is a sectional view of the gastrointestinal tract of a human;

FIG. 2 is a sectional view of a stomach showing an electrostimulation device for delivering tachygastrial electrical stimulation on the antrum of the stomach; and

FIG. 3 illustrates application of an example of a pulse train suitable for producing tachygastrial electrical stimulation.

### DETAILED DESCRIPTION OF THE PREFERRED EMBODIMENT

The present invention provides a process for treating obesity and/or related motor disorders by providing an electrostimulation or pacemaker device attached to, or adjacent to, the stomach, such that the stomach may be electrically stimulated. Alternatively, the electrostimulation or pacemaker device may be attached to, or adjacent to, another part of the gastrointestinal tract such that the portion of the gastrointestinal tract, such as the small intestines or lower intestines, may be electrically stimulated.

The process of the present invention involves treatment of obesity and other syndromes related to motor disorders of the stomach of a patient. The process comprises artificially altering, using sequential electrical pulses for preset periods of time directed to the stomach, thereby decreasing food intake while increasing the patient's feeling of satiety. Electrostimulation of the stomach may also prevent or slow down stomach emptying, thereby slowing food transit through the digestive system, and contributing to the feeling of satiety in the patient. More specifically, the gastric electrical stimulation of the present invention overrides the physiological gastric slow waves and induces tachygastria, an electrical disrhythmia of the stomach that is known to inhibit gastric motility. As such, the electrical stimulation of the present invention comprises tachygastrial electrical stimulation. Accordingly, this method of electrical stimulation inhibits gastric motility and delays the emptying of the stomach, leading to a reduction in food intake and to weight loss.

The tachygastrial electrical stimulation method of the present invention inhibits gastric tone (the resistance of the stomach to stretching) and peristalsis (the wave-like contractions of the stomach). In particular, tachygastria is known to cause gastric hypomotility (or the absence of peristalsis). Thus, the tachygastria electrical stimulation method yields gastric distention (i.e., enlargement of the stomach) and delayed gastric emptying. Gastric distention leads to a feeling of satiety in the patient via gastric stretch receptors, as well as a reduction in gastric accommodation. Likewise, delayed gastric emptying causes an increased and prolonged feeling of stomach fullness, which generally increases the interval between the patient's meals. These effects, in combination, result in a reduction in food intake and weight loss, thus resulting in the treatment of obesity:

According to the present invention, the frequency of a patient's gastric slow waves may be measured using cutaneous electrogastrography (EGG). Tachygastrial electrical stimulation is then preferably performed at a frequency that is at least 30 percent higher than the frequency of the patient's gastric slow wave as measured by the EGG. The tachygastrial electrical stimulation is composed of repeated long pulses having a pulse width of about 50 to about 500 milliseconds and having an amplitude of about 1 to about 20 milliamperes. The stimulation electrodes may be placed anywhere on the stomach, but are preferably attached to the stomach at the antrum or corpus along the greater curvature and/or lesser curvature. If desired, the stimulation electrodes may be placed on, or adjacent to, the small intestines or other visceral organs which interact (e.g., through positive or negative feedback) with the stomach.

For example, the process of this invention may employ tachygastrial electrical stimulation of the stomach at a rate of about 2 to about 30 pulses per minute with each pulse lasting about 50 to about 500 milliseconds, such that there is a pause of about 3 to about 30 seconds between the pulses. Preferably, the individual pulses are at a rate which is at least 30 percent higher than the patient's normal gastric slow waves. The pulse amplitude of the electrostimulation pulses is about 1 to about 20 milliamperes, preferably about 2 to about 15 milliamperes, and the pulse voltage is about 1 to about 10 volts. The tachygastrial electrical stimulation may be delivered with either constant current or constant voltage. These parameters can be varied within these ranges over time (e.g., on a weekly, monthly, or longer basis) in order to prevent, or reducing the risk of, the patient becoming accustomed or acclimatized to the electrostimulation, and thus becoming less responsive or even non-responsive to the electrostimulation.

The method of this invention provides tachygastrial electrical stimulation to the stomach or other visceral organs within the abdominal cavity and/or related to the stomach. The electrostimulation can be applied to more than one location (e.g., two location on the stomach; one location on the stomach and one on the small intestines, and the like). The electrical stimulus preferably consists of a series of single pulses. Generally, the pluses have relatively long durations, preferably about 50 to about 500 milliseconds. The frequency of the stimulation may be slightly higher than the frequency of gastrointestinal slow waves. Preferably, the frequency of the stimulation is at least approximately 30 percent higher than the patient's normal gastric slow wave. More preferably, the frequency of the electrical stimulation is sufficient to induce tachygastria.

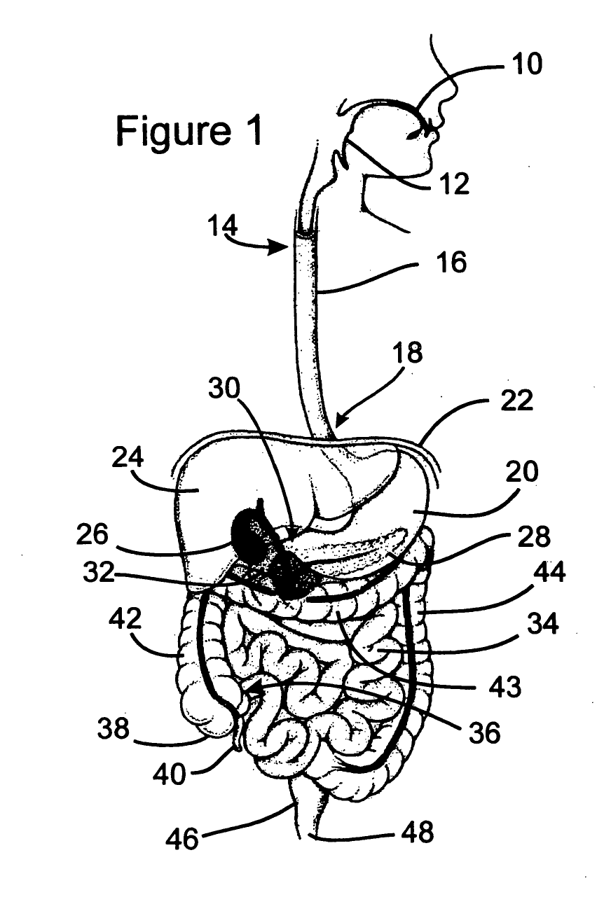

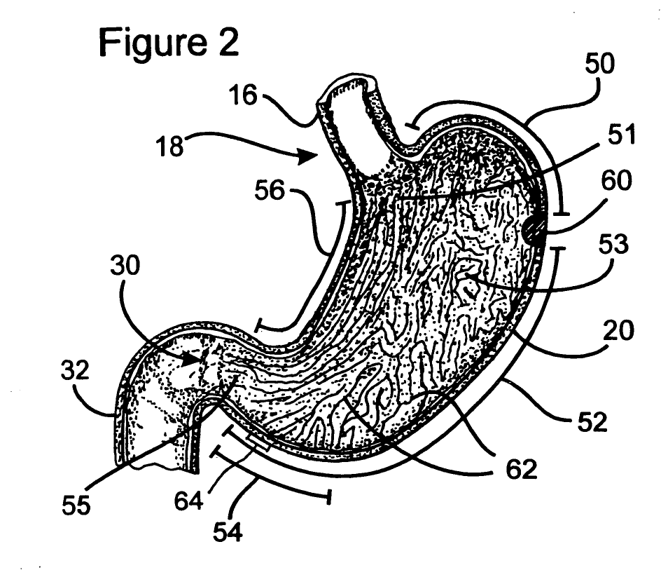

In order to further clarify the process and device for treating obesity and syndromes related to motor disorders of the stomach of a patient, according to the invention, the motor physiology of the gastric viscus is briefly described. As shown in FIGS. 1 and 2, food enters the digestive tract through the mouth 10, passes by the pharynx 12, past the upper esophageal sphincter 14 into the esophagus 16, and then through the lower esophageal sphincter 18 into the stomach 20. As shown in more detail in FIG. 2, the stomach 20 has the fundus ventriculi 50, the cardia 51, the body or corpus ventriculi 53, the greater curvature 52, the lesser curvature 56, the antrum 54, the pylorus 55, the pyloric sphincter 30, the duodenum 32, and mucous folds or rugae 62. The stomach 20 is generally divided into two parts as regards its motility: the fundus ventriculi 50, which has tonic wall movements, and the central part or corpus 53, which is characterized by phasic activity. Propulsive gastric movements begin at a point proximate to the greater curvature 53 which is not clearly identified anatomically and is termed “gastric pacemaker” 60. The gastric pacemaker 60 sends electrical pulses (depolarization potential) at a rate of approximately three times per minute (3 cpm) which spread in an anterograde direction along the entire stomach in the form of waves which have a general sinusoidal shape. The diaphragm 22, liver 24, gall bladder 26, and pancreas 28 are also shown in FIG. 1.

The antrum 54 of the stomach 20 has a continuous phasic activity which has the purpose of mixing the food which is present in the stomach 20. The passage of food into the duodenum 32 is the result of a motility coordinated among the antrum 54, pylorus 55, pyloric sphincter 30, and duodenum 32. The gastric pacemaker 60 spontaneously and naturally generates sinusoidal waves along the entire stomach 20. These waves allow the antrum 54, in coordination with the pylorus 55, the pyloric sphincter 30, and duodenum 32, to allow food to pass into the subsequent portions of the alimentary canal (i.e., small intestines 34 and large intestines, which generally consisting of ascending colon 42, transverse colon 43, and descending colon 34).

The stomach 20 releases food into the duodenum 32, the first part of the small intestines 34, where pancreatic enzymes from the pancreas 28 and bile from the liver 24 are received to aid in digestion and absorption. Food then passes through the small intestines 20 where fats and other nutrients are absorbed. The small intestines generally consist of the duodenum 32, jejunum, and ileum. After passage through the small intestines 20, the generally fluid contents pass through the ileocecal sphincter 36 into the cecum 38 with attached appendix 40. The contents then pass through the ascending colon 42, the transverse colon 43, and descending colon 44; finally, feces pass into the rectum or anal canal 46 for elimination through the anus 48.

Now that the known physiology of the gastric motility of a mammal, such as a human being, has been established, the process according to the invention consists in artificially altering, by means of electrical pulses, the natural gastric motility of a patient by electrostimulation of the stomach. Preferably the electrical pulses are sequential and for preset periods of time. More particularly, the sequential electrical pulses are generated by an implanted electrical stimulator 64 which is applied by laparoscopic means to a portion of, or adjacent to, the stomach. Preferred locations for electrostimulation include the stomach, and more preferably at the antrum or corpus along the greater and/or lesser curvatures of the stomach. Of course, other portions of the gastrointestinal tract can be electrically stimulated using the method of this invention.

The pulse generator of the stimulator 64 can be programmed both for continuous stimulation and for “on demand” stimulation (i.e., at the onset of a particular electrical activity which can be detected by the stimulator 64 itself through the electrocatheter (if modified to monitor electrical activity) or under the control of the patient or medical personnel). The pulse generator preferably includes programmable output variables, wherein variables such as pulse frequency, pulse width, current, and voltage may be programmed into the pulse generator.

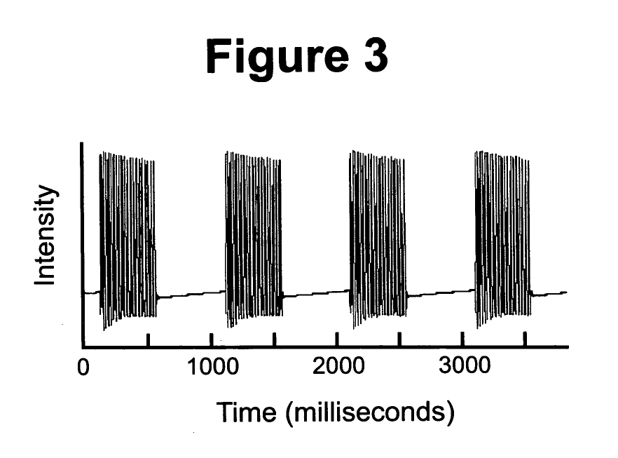

The electrical stimulator 64 preferably has a preset operating frequency and period which may obviously vary according to the alteration of stomach motility to be obtained and/or to the pathological condition of the patient. A typical pulse train suitable for use in the present invention is shown in FIG. 3. Preferably, the desired pulse frequency may be programmed into the pulse generator of the electrical stimulator 64 once it has been determined, such that the same pulse generator may be used with patients with different desired pulse frequencies. Generally, the electrical stimulator 64 has an operating frequency of about 2 to about 30 pulses per minute. Preferably, the process of this invention employs stimulation of the stomach at a rate of about 2 to about 30 pulses per minute with each pulse lasting about 50 to about 500 milliseconds, such that there is a pause of about 3 to about 30 seconds between the pulses. More preferably, the pulse rate is about 30 percent higher than the normal gastric pulse rate, with each pulse lasting about 50 to about 500 milliseconds. The electrical stimulation may be delivered with either constant voltage or constant current. The electrical discharge of each pulse can vary from approximately 1 to approximately 15 volts, and more preferably about 2 to about 5 volts, for voltage-controlled stimulation and from 2 to 15 milliamperes for constant current stimulation.

The present invention generally uses conventional laparoscopic or minimally invasive surgical techniques to place the desired electrostimulation device or devices on, or adjacent to, the stomach or other portions of the gastrointestinal tract, whereby electrostimulation of the stomach or gastrointestinal tract can be effected. Conventional electrostimulation devices may be used in the practice of this invention. Such devices include, for example, those described in U.S. Pat. No. 5,423,872 (Jun. 3, 1995); U.S. Pat. No. 5,690,691 (Nov. 25, 1997); U.S. Pat. No. 5,836,994 (Nov. 17, 1998); U.S. Pat. No. 5,861,014 (Jan. 19, 1999); PCT Application Serial No. PCT/US98/10402 (filed May 21, 1998) and U.S. patent application Ser. No. 09/424,324 (filed Jan. 26, 2000); U.S. Pat. No. 6,041,258 (Mar. 21, 2000); U.S. patent application Ser. No. 09/640,201 (filed Aug. 16, 2000); PCT Application Serial No. PCT/US00/09910 (filed Apr. 14, 2000) based on U.S. Provisional Application Ser. Nos. 60/129,198 and 60/129,199 (both filed Apr. 14, 1999); PCT Application Serial No. PCT/US00/10154 (filed Apr. 14, 2000) based on U.S. Provisional Application Ser. Nos. 60/129,209 (filed Apr. 14, 1999) and 60/466,387 (filed Dec. 17, 1999); and U.S. Provisional Patent Application Ser. No. 60/235,660 (filed Sep. 26, 2000). All of these patents, patent applications, provisional patent applications, and/or publications, as well as all such references cited in the present specification, are hereby incorporated by reference in their entireties.

Preferred electrostimulation devices include electrocatheters having an elongated body with a distal end having an electrostimulation lead or leads mounted on, or attached to, the gastrointestinal tract and a proximal end for attachment to a pulse generator. The pulse generator preferably includes programmable output variables, wherein variables such as pulse frequency, pulse width, current, and voltage may be programmed into the pulse generator. The electrostimulation lead or leads are attached to a power source through, or with, the pulse generator. The power source preferably includes a rechargeable battery, but alternative power sources may also be used. Such preferred electrostimulation devices are described in, for example, PCT Application Serial Number PCT/US98/10402 (filed May 21, 1998), U.S. patent application Ser. No. 09/424,324 (filed Jan. 26, 2000), and U.S. patent application Ser. No. 09/640,201 (filed Aug. 16, 2000). Of course, care should be taken in placement or attachment of the electrostimulation device to avoid physical damage to the gastrointestinal tract.

The present methods can also be used in combination with electrostimulation of other parts of the gastrointestinal tract. For example, electrostimulation could be applied to several locations within the gastrointestinal tract, such as two electrodes on the stomach or one electode on the stomach and another on the small intestines. The sites of electrostimulation could be phased or non-phased in relation to one another.

Preferably, the electrostimulation device is an implantable device. However, the electrostimulation device may also be an external device if such a device is desirable.

The present methods can also use a sensor or sensors to detect food entering the stomach, initiation of digestive processes, and/or other process or events associated with digestion within, or related to, the stomach to begin the stimulation for a predetermined time. Such sensors and processes using such sensors are described in detail in our copending Provisional Application Ser. No. 60/557,736, filed on the same date as the present application and entitled “Sensor Based Gastrointestinal Electrical Stimulation for the Treatment of Obesity or Motility Disorders” (Docket 79775), which is incorporated by reference in its entirety.

### EXAMPLE 1

### Tachygastrial Electrical Stimulation-Induced Tachygastria in Dogs

The aim of this study was to investigate whether tachygastrial electrical stimulation was capable of inducing tachygastria. The study was performed on six healthy female dogs. The dogs were chronically implanted with 3 pairs of electrodes on the gastric serosa along the greater curvature of the stomach. One distal pair, which was mounted 3-6 cm above the pylorus, was used for electrical stimulation. The other two pairs were about 4 and 8 cm, respectively, above the pair used for stimulation.

Each study session consisted of nine stimulation periods. After a 30 minute baseline recording (i.e., no electrical stimulation applied), tachygastrial electrical stimulation with a pulse width of 100 milliseconds and a pulse amplitude of 6 milliamperes was initiated. Four different frequencies (i.e., 7 cycles per minute (cpm), 9 cpm, 14 cpm, and 18 cpm) were tested, with each frequency used in a separate stimulation period. Each stimulation period lasted for 20 minutes and was followed by a 20 minute recovery period. Gastric slow waves were recorded from the two proximal pairs of electrodes. Spectral analysis was performed to calculate the percentage of normal 4-6 cpm slow waves or tachygastria (greater than 6 cpm).

It was found that tachygastrial electrical stimulation at 7 cpm and 14 cpm induced complete entrainment (i.e., the gastric slow waves were phase-locked with a stimuli at a frequency of 7 cpm). The percentage of entrainment time was approximately 64.5 percent (±3.5%) with tachygastrial electrical stimulation at 7 cpm and 53.2 percent (±5.9%) with tachygastrial electrical stimulation at 14 cpm. No complete entrainment was found during tachygastrial electrical stimulation at 9 cpm and 18 cpm.

It was also found that tachygastrial electrical stimulation at tachygastrial frequencies significantly reduced the percentage of normal slow waves and induced tachygastria. The percent of normal slow waves and percent tachygastria at each tachygastrial electrical stimulation frequency is shown in the following table:

| Stimulation | | |
|---|---|---|
| Frequency | Normal Slow | |
| (cpm) | Waves (%) | Tachygastria (%) |
| Baseline | 82.4 ± 6.0 | 1.9 ± 1.3 |
| (no stimulation) | | |
| 7 | 13.7 ± 3.8 | 78.1 ± 4.6 |
| 9 | 18.5 ± 10.2 | 52.8 ± 6.2 |
| 14 | 8.3 ± 3.4 | 76.9 ± 6.8 |
| 18 | 12.2 ± 4.8 | 55.6 ± 8.4 |

Thus, significant increase in tachygastria was present when tachygastrial electrical stimulation was applied. Notably, it was found that tachygastrial electrical stimulation at 7 cpm and 14 cpm induced a higher percentage of tachygastria.

### EXAMPLE 2

### Tachygastrial Electrical Stimulation-Inhibited Gastric Motility in Dogs

The aim of this study was to investigate whether tachygastrial electrical stimulation is capable of inhibiting gastric motility. The study was performed in six dogs chronically implanted with one pair of gastric serosal electrodes located 4 cm above the pylorus. A chronic gastric cannula was also in place for the insertion of a manometric catheter into the stomach to measure gastric contractions. The study was performed at least two weeks after the surgical procedure to implant the electrodes and catheter and when the animals were healthy.

At the time of the experiment, each dog was fed one can of dog food. Immediately after eating, antral contractions were measured using a manometric catheter placed in the distal antrum via the gastric cannula. The recording was composed of three 30 minute consecutive postprandial periods: (1) baseline; (2) tachygastrial electrical stimulation; and (3) recovery. Tachygastrial electrical stimulation was performed at a frequency of 9 cpm, a pulse width of 300 milliseconds, and a pulse amplitude of 6 milliamperes. The results of this test, reported in contractions per minute (cpm), are shown in the following table:

| | Baseline | Stimulation | Recovery |
|---|---|---|---|
| Dog | (cpm) | (cpm) | (cpm) |
| 1 | 3.9 | 0 | 1.52 |
| 2 | 4.5 | 0.4 | 2.1 |
| 3 | 4.7 | 0.1 | 0.2 |
| 4 | 5.2 | 0 | 2.4 |
| 5 | 4.5 | 0.1 | 1.6 |
| 6 | 4.6 | 0.05 | 3.6 |
| Average | 4.57 | 0.11 | 1.90 |

A substantial decrease in the number of contractions per minute was demonstrated when tachygastrial electrical stimulation was applied. Thus, it is evident that tachygastrial electrical stimulation is capable of effectively inhibiting gastric contractions.

### EXAMPLE 3

### Tachygastrial Electrical Stimulation-Induced Distention and Reduction in Gastric Accommodation in Dogs

This study was completed to determine whether tachygastrial electrical stimulation could induce gastric distention and reduce gastric accommodation. The study was performed on five healthy dogs that ranged in weight from 17 to 25 kilograms. The dogs were implanted with a gastric cannula and one pair of electrodes along the greater curvature of the stomach, 4 cm above the pylorus. Barostat studies were conducted in overnight fasted, conscious animals. A polyethylene balloon (700 milliliters volume, 10 centimeters in diameter) was introduced into the stomach via the gastric cannula and implanted on the anterior side of the stomach, about 10 cm above the pylorus.

The gastric volume was recorded under a constant minimal pressure for 30 inutes at baseline, 30 minutes with tachygastrial electrical stimulation, and 60 minutes after a liquid meal of Boost® (237 milliliters, 240 kcal) with tachygastrial electrical stimulation. Tachygastrial electrical stimulation was performed at a frequency of 9 cpm, a pulse width of 200 milliseconds, and a pulse amplitude of 6 milliamperes. In the control session performed on a separate day, gastric tone was recorded for 30 minutes at baseline and 60 minutes after the same Boost® test meal (but tachygastrial electrical stimulation was not performed).

It was found that tachygastrial electrical stimulation consistently increased the intra-gastric balloon volume in all the tested animals. The mean fasting gastric volume was increased from a baseline value of 104.6±44.2 milliliters to 308.8±42.4 milliliters during tachygastrial electrical stimulation and 453.8±44.2 milliliters after the meal.

Additionally, in comparison with the control session, the gastric accommodation (i.e., the volume difference between pre- and post-meal) was significantly reduced with tachygastrial electrical stimulation. That is, without tachygastrial electrical stimulation gastric accommodation was 267.1±28.9 milliliters, while with tachygastrial electrical stimulation the gastric accommodation fell to 145.1±24.3 milliliters. The postprandial (post-meal) volume of the stomach did not show any difference with or without tachygastrial electrical stimulation.

### EXAMPLE 4

### Tachygastrial Electrical Stimulation-Delayed Gastric Emptying in Dogs

This study was undertaken to investigate the effect of tachygastrial electrical stimulation on gastric emptying and acute food intake. The study was performed in six healthy female hound dogs having weights of about 22.5 to about 27.5 kilograms. The dogs were chronically implanted with 4 pairs of electrodes on the gastric serosa and equipped with a duodenal cannula for the assessment of gastric emptying.

The study was composed of 2 separate experiments. The first experiment was designed to study the effect of tachygastrial electrical stimulation on food intake, water intake, and signs and symptoms, and was composed of two sessions conducted on two different days. After a 28 hour fast, stimulation or no stimulation was initiated, depending upon if the test was with tachygastrial electrical stimulation or the control test, and 30 minutes later the dogs were given unlimited solid food and water for 60 minutes either with or without stimulation. The results of the first experiment are shown in the following table:

| Time (minutes) after | Gastric Emptying | Gastric Emptying with |
|---|---|---|
| Start of Experiment | without Stimulation (%) | Stimulation (%) |
| 30 | 36.1 ± 7.9 | 28.5 ± 7.5 |
| 45 | 47.7 ± 7.5 | 37.1 ± 7.6 |
| 60 | 54.6 ± 8.1 | 43.8 ± 8.6 |
| 75 | 57.9 ± 7.3 | 47.8 ± 8.2 |
| 90 | 62.1 ± 6.2 | 51.5 ± 7.5 |

For this first experiment, it was found that tachygastrial electrical stimulation resulted in a significant reduction in food intake in the second experiment, but had no significant effect on water intake. The mean food intake with tachygastrial electrical stimulation was 227.3±38.6 g, in comparison with 317.6±27.5 g without electrical stimulation.

The second experiment was designed to study the effect of tachygastrial electrical stimulation on gastric emptying and was composed of 2 sessions in a random order, with at least a 72 hour lapse between the two sessions. The dogs were fasted overnight before the study. Thirty minutes after the dog was put into a restraining sling, either no stimulation or tachygastrial electrical stimulation, according to the test being conducted, was initiated and 30 minutes later the dog was fed with 237 milliliters of Ensure® mixed with 100 milligrams phenol red. Thereafter, no stimulation or tachygastrial electrical stimulation was continuously applied for another 90 minutes and gastric emptying chyme was collected every 15 minutes for 90 minutes. Tachygastrial electrical stimulation was fixed at a tachygastrial frequency of 9 cycles per minute (cpm) with a pulse width of 100 milliseconds and a pulse amplitude of 2 milliamperes. The electrical stimulation was applied through an electrode attached to the stomach 6 cm above the pylorus.

In both experiments, tachygastrial electrical stimulation did not induce any remarkable signs or symptoms in comparison with the baseline session.

### EXAMPLE 5

### Tachygastrial Electrical Stimulation-Reduced Food Intake in Dogs

A short-term food intake study was performed in five dogs that were chronically implanted with a pair of electrodes on the gastric serosa, 4 cm above the pylorus. The connecting wires were brought out to the abdominal skin subcutaneously and protected with a jacket and collar. After a complete recovery from surgery (three weeks), the dogs were fed with unlimited food each day between 9:00 am and 11:00 am for three weeks. No food was given at other times during those three weeks. Water was also provided ad libitum. This schedule was used to acclimate the dogs to eating food during a set period of time each day.

During the fourth week, tachygastrial electrical stimulation was performed via a portable stimulator that was attached to the back of the dogs from 8:30 am to 11:00 am. The tachygastrial electrical stimulation was applied at a frequency of 9 cpm, a pulse width of 100 milliseconds, and a pulse amplitude of 6 milliamperes. The animals were then given unlimited regular solid food from 9:00 am to 11:00 am, as in the preceding three weeks. At 11:00 am, the leftover food was removed and the amount of food intake was recorded.

During the fifth week, the same procedure was followed, but the portable stimulator was not turned on and, therefore, the tachygastrial electrical stimulation was not applied. The average daily food intake during the fifth week was then compared with that of the fourth week, during which tachygastrial electrical stimulation was applied.

The average daily food intake was found to be 517±18 grams during the control week (fifth week) and 422±22 grams during the week in which tachygastrial electrical stimulation was applied (fourth week). Thus, it was determined that the application of tachygastrial electrical stimulation resulted in a reduction in food intake of approximately twenty percent.

### EXAMPLE 6

### Tachygastrial Electrical Stimulation-Induced Satiety in Human Patients with Obesity

In order to investigate whether tachygastrial electrical stimulation was able to induce satiety in human patients, a study was performed in eight female obese patients, each of which had a body mass index between about 35 and about 38 kg/m². The patients were scheduled for a laparoscopic procedure other than in connection with this study, but consented to the placement of two pairs of stainless steel temporary cardiac pacing wires on the gastric serosa during the same laparoscopic procedure. One pair of electrodes was placed 6 cm above the pylorus along the greater curvature and the other was placed 10 cm above the pylorus. The distance between the two electrodes in each pair was about 1 cm. The electrodes were imbedded in the seromuscular layer without any suture and were removed by lightly pulling the electrodes from the patient at the end of the study. The connection wires were brought out to the abdomen subcutaneously and protected with sterilized gauze.

The experiment was performed two weeks after the placement of the electrodes in the hospital. The protocol comprised of a fasting session and two meal sessions (lunch and dinner) in one day. In the fasting session, a baseline recording of the gastric slow wave was made for 30 minutes via both pairs of implanted gastric electrodes. After this, tachygastrial electrical stimulation was applied through the distal pair of the electrodes using different stimulation parameters. The parameters that were varied were the pulse width, which was increased from about 100 milliseconds to about 500 milliseconds, and the stimulation frequency, which was varied between about 7 cpm and about ˜12 cpm.

In the first four patients, a portable stimulator was used and the output was fixed at 6 milliamperes, which was the maximum output of the device. A second device which had a higher output was used for the second set of four patients and the output was increased to 10 milliamperes if no noticeable effects in the patient were observed. There was a period of time of about 5 minutes (if no effects were noted by the patient in connection with the prior tachygastrial electrical stimulation) to 30 minutes (if the prior tachygastrial electrical stimulation caused effects which were noted or reported by the patient) during which tachygastrial electrical stimulation was not performed between the two consecutive stimulation sessions.

The patients were not told whether or not tachygastrial electrical stimulation was performed, but were asked to report any symptoms or feelings, including satiety, bloating or fullness of the stomach, nausea, vomiting, and pain. The two meal-related sessions were composed of a sham-tachygastrial electrical stimulation session in which no tachygastrial electrical stimulation was applied and a real tachygastrial electrical stimulation session when tachygastrial electrical stimulation was applied. The order of the two sessions was randomized and the patient did not know whether tachygastrial electrical stimulation was being applied or not. For the tachygastrial electrical stimulation session with the meal, the parameters of the tachygastrial electrical stimulation were set at the most effective values acceptable and tolerable by the patients in the fasting state. The meals were chosen by the patients from the hospital cafeteria and the patients were asked to choose their favorite foods without any restrictions.

Normal gastric slow waves were recorded during a baseline period and found to have a frequency of about 3 cycles per minute (cpm). Tachygastrial electrical stimulation at a frequency of 9 cpm and a pulse width of 300 milliseconds entrained gastric slow waves at 4.5, a tachygastrial frequency (a frequency of more than 4 cpm in humans inhibits gastric motility and causes tachygastria), in all patients.

Tachygastrial electrical stimulation was found to induce satiety in three of the first set of four patients at an output amplitude of 6 milliamperes and in all four patients of the second set of four at an output of between 6 milliaperes and 10 milliamperes. Five of the eight patients felt stomach fullness or bloating. None of the patients felt nausea, vomiting, or pain at the maximum tested output of each group (either 6 milliamperes or 6-10 milliamperes).

All patients reported increased satiety and an increased feeling of stomach fullness with tachygastrial electrical stimulation in comparison with the session without tachygastrial electrical stimulation. Likewise, seven of the eight patients reported a reduced appetite and ate less with tachygastrial electrical stimulation than without tachygastrial electrical stimulation. Two patients stopped eating in the middle of a meal due to their increased satiety.

One patient of the second group had a brief episode of vomiting or spitting when tachygastrial electrical stimulation was applied at the maximum output of 10 milliamperes, but felt comfortable five minutes after the output was turned to a slightly lower level. Another patient reported slight nausea after a meal during which tachygastrial electrical stimulation was applied and regretted eating too much, but refused an offered adjustment to a lower output level. No other dyspeptic symptoms were reported.

This human study showed that tachygastrial electrical stimulation was capable of inducing satiety and stomach fullness in human patients. It was also determined that in some cases, dyspeptic symptoms may be induced by tachygastrial electrical stimulation having a higher output, but that such symptoms could be eliminated by varying the output of the tachygastrial electrical stimulation tachygastrial electrical stimulation. Additionally, the presence of some mild dyspeptic symptoms may be beneficial or desireable in obese patients in that they may serve as a motivation to the patient to modify their eating habits in a beneficial way. Finally, it was noted in this study that it is possible to induce satiety without other dyspeptic symptoms by setting the tachygastrial electrical stimulation parameters at individualized levels.

While the invention has been described in the specification and illustrated in the drawings with reference to preferred embodiments, it will be understood by those skilled in the art that various changes may be made and equivalents may be substituted for elements thereof without departing from the scope of the present invention as defined in the appended claims. In addition, many modifications may be made to adapt a particular situation or material to the teachings of the invention, as defined in the appended claims, without departing from the essential scope thereof. Therefore, it is intended that the present invention not be limited to the particular embodiments illustrated by the drawings and described in the specification as the best modes presently contemplated for carrying out the present invention, but that the present invention will include any embodiments falling within the description of the appended claims.

1. A method for treatment of obesity in a patient, said method comprising
implanting an electrostimulation device comprising at least one electrostimulation lead, a pulse generator, and an electrical connector connecting the at least one electrostimulation lead and the pulse generator, such that the at least one electrostimulation lead is attached to, or adjacent to, the patient's stomach, whereby electrical stimulation from the pulse generator can be provided to the stomach through the at least one electrostimulation lead; and
supplying electrical stimulation to the stomach through the at least one electrostimulation lead;
wherein the electrical stimulation is sufficient to induce tachygastria in the patient and wherein the electrical stimulation has a pulse width of about 50 to about 500 milliseconds.

2. The method of claim 1, wherein the electrical stimulation is supplied at an operating frequency of about 2 to about 30 pulses per minute.

3. The method of claim 2, wherein the operating frequency is about 2 to about 15 pulses per minute.

4. The method of claim 2, wherein the electrical stimulation is supplied at an operating frequency at least about 30 percent higher than the patient's normal gastric slow wave frequency.

5. The method of claim 1, wherein the at least one electrostimulation lead is attached to the stomach along the stomach's greater curvature.

6. The method of claim 1, wherein the at least one electrostimulation lead is attached to the stomach along the stomach's lesser curvature

7. The method of claim 1, wherein the pulse generator includes at least one programmable output variable.

8. The method of claim 7, wherein the at least one programmable output variable includes the pulse width and wherein the pulse width is about 50 to about 500 milliseconds.

9. The method of claim 7, wherein the at least one programmable output variable includes current.

10. The method of claim 7, wherein the at least one programmable output variable includes voltage.

11. The method of claim 1, wherein the electrostimulation device includes a rechargeable battery.

12. The method of claim 1, wherein the electrical stimulation is supplied with constant current.

13. The method of claim 1, wherein the electrical stimulation is supplied with a current of about 1 to about 20 milliamperes.

14. The method of claim 13, wherein the current is about 2 to about 15 milliamperes.

15. The method of claim 14, wherein the current is about 5 to about 10 milliamperes.

16. The method of claim 1, wherein the electrical stimulation is delivered with constant voltage.

17. The method of claim 1, wherein the electrical stimulation is delivered with a voltage of about 1 to about 10 volts.

18. The method of claim 1, wherein the at lead one electrostimulation lead comprises at least one electrode.

19. The method of claim 18, wherein the at least one electrode comprises two electrodes.

20. A method for treatment of obesity in a patient, said method comprising
implanting at least two electrostimulation devices, wherein each of the electrostimulation devices comprises at least one electrostimulation lead and an electrical connector for attachment to a pulse generator, such that the at least one electrostimulation lead is attached to, or adjacent to, the patient's gastrointestinal tract, whereby electrical stimulation can be provided to the gastrointestinal tract through the at least one electrostimulation lead at two or more different locations along the gastrointestinal tract; and
supplying electrical stimulation to the gastrointestinal tract through the at least one electrostimulation lead of the two electrostimulation devices at two or more difference locations along the gastrointestinal tract;
wherein the operating frequency of the electrical stimulation is sufficient to induce tachygastria in the patient and wherein the electrical stimulation has a pulse width of about 50 to about 500 milliseconds.

21. The method of claim 20, wherein the operating frequency is about 2 to about 30 pulses per minute.

22. The method of claim 21, wherein the operating frequency is about 2 to about 15 pulses per minute.

23. The method of claim 20, wherein the operating frequency is at least about 30 percent higher than the patient's normal gastric slow wave frequency

24. The method of claim 20, wherein the electrical stimulation is delivered with a current of about 1 to about 20 milliamperes.

25. The method of claim 24, wherein the electrical stimulation is delivered with a current of about 2 to about 15 milliamperes.

26. The method of claim 25, wherein the electrical stimulation is delivered with a current of about 5 to about 10 milliamperes.

27. The method of claim 12, wherein the electrical stimulation is delivered with a voltage of about 1 to about 10 volts.

28. An electrostimulation device for the treatment of obesity in a patient, said electrostimulation device comprising at least one electrostimulation lead, a pulse generator, and an electrical connector connecting the at least one electrostimulation lead and the pulse generator, such that the at least one electrostimulation lead is attached to, or adjacent to, the patient's stomach, whereby electrical stimulation from the pulse generator can be provided to the stomach through the at least one electrostimulation lead; wherein the electrical stimulation is capable of inducing tachygastria in the patient, wherein the electrical pulse generator includes at least one programmable output variable, wherein the at least one programmable output variable is pulse width, and wherein the electrical stimulation has a pulse width of about 50 to about 500 milliseconds.

29. The electrostimulation device according to claim 28, wherein the at least one programmable output variable further comprises electrical stimulation frequency.

30. The electrostimulation device according to claim 29, wherein the at least one programmable output variable further comprises current.

31. The electrostimulation device according to claim 29, wherein the at least one programmable output variable further comprises voltage.

32. The electrostimulation device according to claim 28, wherein the electrostimulation device includes a rechargeable battery.

33. A method for treatment of obesity in a patient, said method comprising
implanting an electrostimulation device comprising at least one electrostimulation lead, a pulse generator, and an electrical connector connecting the at least one electrostimulation lead and the pulse generator, such that the at least one electrostimulation lead is attached to, or adjacent to, the patient's stomach, whereby electrical stimulation from the pulse generator can be provided to the stomach through the at least one electrostimulation lead; and
supplying electrical stimulation to the stomach through the at least one electrostimulation lead;
wherein the electrical stimulation has a pulse width of about 50 to about 500 milliseconds and an operating frequency at least about 30 percent higher than the patient's normal gastric slow wave frequency.

34. The method of claim 33, wherein the at least one electrostimulation lead is attached to the stomach along the stomach's greater curvature.

35. The method of claim 33, wherein the at least one electrostimulation lead is attached to the stomach along the stomach's lesser curvature

36. The method of claim 33, wherein the pulse generator includes at least one programmable output variable.

37. The method of claim 36, wherein the at least one programmable output variable includes the pulse width.

38. The method of claim 36, wherein the at least one programmable output variable includes current.

39. The method of claim 36, wherein the at least one programmable output variable includes voltage.

40. The method of claim 36, wherein the electrostimulation device includes a rechargeable battery.

41. The method of claim 36, wherein the electrical stimulation is supplied with constant current.

42. The method of claim 36, wherein the electrical stimulation is supplied with a current of about 1 to about 20 milliamperes.

43. The method of claim 42, wherein the current is about 2 to about 15 milliamperes.

44. The method of claim 42, wherein the current is about 5 to about 10 milliamperes.

45. The method of claim 33, wherein the electrical stimulation is delivered with constant voltage.

46. The method of claim 45, wherein the electrical stimulation is delivered with a voltage of about 1 to about 10 volts.

47. The method of claim 33, wherein the at lead one electrostimulation lead comprises at least one electrode.

48. The method of claim 33, wherein the at lead one electrostimulation lead comprises two electrodes.

49. The method of claim 33, wherein the electrostimulation is sufficient to induce tachygastria in the patient.

50. The method of claim 34, wherein the electrostimulation is sufficient to induce tachygastria in the patient.

51. The method of claim 35, wherein the electrostimulation is sufficient to induce tachygastria in the patient.
